(19) United States
(12) Patent Application Publication
(10) Pub. No.: US 2025/0328942 A1
(43) Pub. Date: Oct. 23, 2025

(54) ORTHOTIC APPARATUS AND METHOD

(71) Applicants: Graeme White, Kitchener (CA); Moona Rahemtulla, Kitchener (CA)

(72) Inventors: Graeme White, Kitchener (CA); Moona Rahemtulla, Kitchener (CA)

(21) Appl. No.: 18/638,902

(22) Filed: Apr. 18, 2024

(51) Int. Cl. G06Q 30/0601; A61F 5/14; G16H 10/60

(52) U.S. Cl. CPC G06Q 30/0631; G06Q 30/0641; G16H 10/60; A61F 5/14

(57) ABSTRACT

An orthotic apparatus and method that is operable to provide foot mapping of a user and provide guidance to an orthotic insert that mateably fits the user's foot. The present invention includes a kiosk wherein the kiosk includes an upright housing and a base member operably coupled thereto. The base member has a foot plate member thereon configured to have a user superpose their feet onto the upper surface thereof. The method of the present invention includes a plurality of steps that require the user to manipulate the position of their feet and further place increased pressure on various parts thereof. During manipulation of the feet the multitude of sensors embedded within the foot plate member provide surface data mapping of the bottom of the user's foot. The data mapping obtains a plurality of alternate measurements of three regions of the user's foot to provide guidance on a proper orthotic.

### FIELD OF THE INVENTION

The present invention relates generally to footwear, more specifically but not by way of limitation, an apparatus and method configured to be employed by a user wherein the apparatus is configured to capture a complete scan of a bottom of a user's foot and utilize the scanned data to guide purchase of an orthotic insert for the user.

### BACKGROUND

Millions of people suffer from foot pain that is caused by improper fitting shoes. An improper fit for shoes can create numerous types of maladies and pain with the feet and will persist unless a proper shoe and/or orthotic is introduced. The human foot has twenty-six bones, thirty joints, and hundreds of muscles, ligaments, and tendons. Each foot has over seven thousand nerve endings that relay needed information like surface stability, temperature, and slope. These elements work together to allow us to make complex movements needed for walking, standing, and keeping our balance. One of the many problems that can occur as a result of improper fitting shoes is plantar fasciitis. Plantar fasciitis is caused by the inflammation of the plantar fascia, a strong band of tissue running from the heel to the ball of the foot. Over two million people experience plantar fasciitis the most common cause of heel pain. Inflammation occurs from overuse, standing on a hard surface for too long, age, and wearing unsupportive shoes like flip flops or overly flexible flats or sneakers.

Many other foot problems occur from wearing shoes that lack proper support. Conditions such as but not limited to claw toes and hammertoes can be caused by wearing a shoe that does not fit properly or lacks the proper support. Chances of slips, trips, falls, and missteps are increased when wearing improper footwear. In addition, any fracture can happen, especially in ill-fitting shoes. Damage to the ligaments and tendons of the foot and ankle, strains and sprains, happen when shoes lack proper fit.

Accordingly, there is a need for an apparatus and method that is operable to provide a scan of a user's foot in order to capture the measurements of the medial longitudinal arch, lateral longitudinal arch, and transverse arch so as to provide an orthotic insert capable of being utilized by individuals having low, normal and high arches.

### SUMMARY OF THE INVENTION

It is the object of the present invention to provide a method operable to provide an output of a proper orthotic insert wherein the method of the present invention employs a kiosk having a foot plate member configured to provide two dimensional and three dimensional scan data of a user's foot.

Another object of the present invention is to provide an orthotic apparatus and method thereof wherein the method of the present invention includes requiring the user to manipulate and reposition their feet on the foot plate member of the kiosk of the present invention.

A further object of the present invention is to provide a method operable to provide an output of a proper orthotic insert wherein the present invention includes providing of four alternate sizes for each of the three arch heights for a user's foot.

Yet a further object of the present invention is to provide an orthotic apparatus and method thereof wherein the orthotic of the present invention includes support rib members integrally formed therein.

Still another object of the present invention is to provide a method operable to provide an output of a proper orthotic insert wherein the method of the present invention further includes requiring a user to shift their weight and lift their feet during the data acquisition process.

An additional object of the present invention is to provide an orthotic apparatus and method thereof wherein some embodiments of the orthotic of the present invention include an aperture adjacent the heel of the user's foot.

Yet a further object of the present invention is to provide a method operable to provide an output of a proper orthotic insert wherein based on the data acquired for the user' foot the method will suggest one orthotic from an available database of fourteen orthotics for the user.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is a perspective view of an exemplary kiosk of the present invention; and

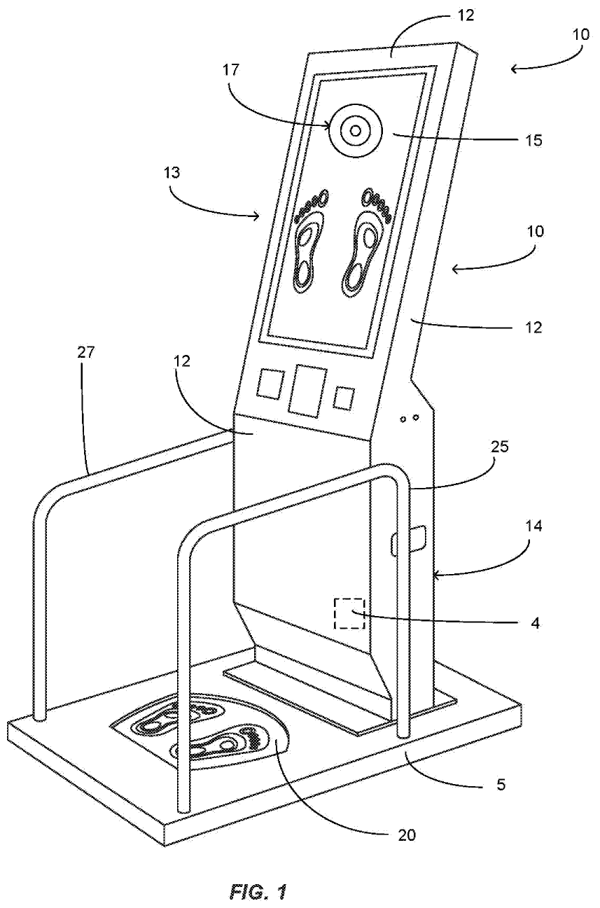

FIG. 2 is a side perspective view of an exemplary orthotic of the present invention; and

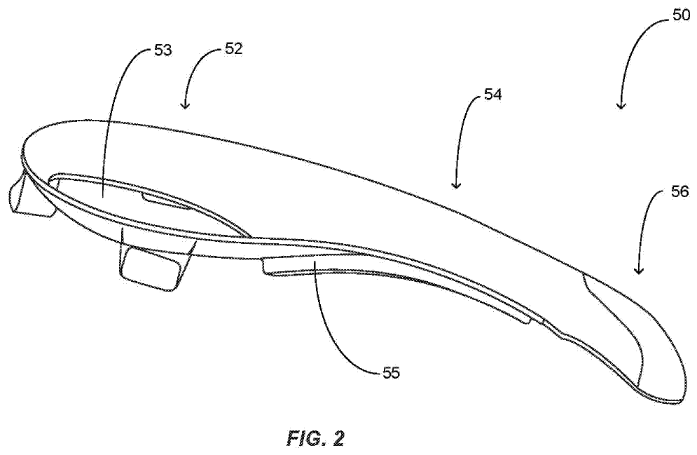

FIG. 3 is an outline of the method of the present invention.

### DETAILED DESCRIPTION

Referring now to the drawings submitted herewith, wherein various elements depicted therein are not necessarily drawn to scale and wherein through the views and figures like elements are referenced with identical reference numerals, there is illustrated an orthotic apparatus and method 100 constructed according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

Referring in particular to the Figures submitted herewith, the orthotic apparatus and method 100 employs the utilization of a kiosk 10 that is configured to provide data acquisition of a user's feet so as to guide a user to a proper exemplary orthotic 50. It should be understood within the scope of the present invention that the kiosk 10 could be provided in various alternate embodiments in addition to the kiosk 10 discussed herein. Alternate embodiments and elements of that are contemplated within the scope of the present invention include elements such as but not limited to shelving for storage of orthotics 50. The kiosk 10 includes a housing 12 that is manufactured from a durable material such as but not limited to plastic or metal. The housing 12 comprises a plurality of walls that are integrally formed creating an upper portion 13 and lower portion 14.

Disposed in the upper portion 13 is a graphical display unit 15. The graphical display unit 15 is a conventional LCD display unit that is configured to provide information to the user of the orthotic apparatus and method 100 during use thereof. By way of example but not limitation, the graphical display unit 15 is operable to provide a graphical map of the user's feet during the mapping process thereof. It should be understood within the scope of the present invention that the graphical display unit 15 could be provided in alternate sizes. The graphical display unit 15 provides a status ring indicator 17 to the user during execution of the method of the orthotic apparatus and method 100. The status ring indicator 17 provides a real time update on the data mapping of the foot of the user.

The kiosk 10 includes foot plate member 20 wherein the foot plate member 20 is operably mounted within the base member 5. The foot plate member 20 includes a multitude of sensors that are configured to provide the mapping of the user's feet during the execution of the method of the orthotic apparatus and method 100. In the preferred embodiment of the present invention, the foot plate member 20 includes at least two sensors for every square centimeter of the foot plate member 20. Mounted to the base member 5 extending upward therefrom are side rail members 25,27. The side rail members 25, 27 are mounted utilizing suitable durable techniques and are manufactured from a rigid material such as but not limited to metal. The side rail members 25,27 provide an element for a user to engage and support themselves during execution of the method of the orthotic apparatus and method 100. It is contemplated within the scope of the present invention that the side rail members 25, 27 could be provided in alternate sizes and shapes.

The orthotic apparatus and method 100 provides an inventory of fourteen premanufactured orthotics 50 wherein one of which is suggested to a user subsequent execution of the method of the orthotic apparatus and method 100. The orthotic 50 is manufactured from suitable support materials such as but not limited to foam rubber, gel and plastic. The orthotic 50 includes a hindfoot region 52, a midfoot region 54 and a forefoot region 56. Each of the aforementioned regions are manufactured with alternate measurements and dimensions within the inventory of the fourteen provided orthotics 50 in the present invention. While not illustrated herein, it is contemplated within the scope of the present invention that the kiosk 10 could include shelfs so as to provide storage of the inventory of the orthotics 50. The hindfoot region 52 in some embodiments may be provided with a heel aperture 53. The heel aperture 53 is formed utilizing suitable techniques and is bore through the all layers of the orthotic 50. The midfoot region 54 is the area adjacent the arch of the foot of the user. The midfoot region 54 includes support rib members 55 the extend longitudinally along the midfoot region 54. The support rib members 55 are manufactured from a material more rigid that the orthotic and are arcuate in shape to provide support for an arch of a user's foot. The radius of the support rib members 55 vary based of the height of the arch of the user's foot. It should be understood within the scope of the present invention that the support rib members 55 could be provided in alternate quantities, radius and lengths. The orthotics 50 are provided with either no support rib members 55 or a plurality of support rib members 55 wherein the radius of the support rib members will vary so as to accommodate arch heights of low, medium and high.

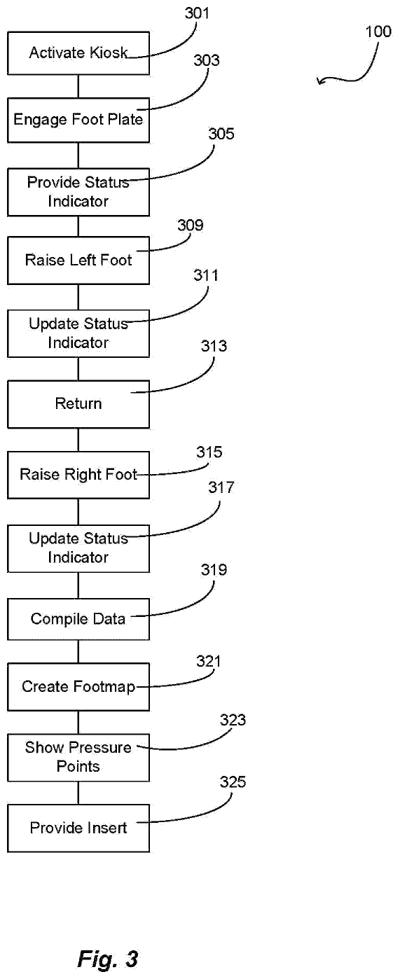

Referring in particular to FIG. 3 submitted as a part hereof, the method of the orthotic apparatus and method 100 is diagrammed therein. In step 301, a user will activate a kiosk 10. It should be understood within the scope of the present invention that the kiosk 10 could be located in various alternate locations such as but not limited to a retail store. Step 303, the user will be directed to engage the foot plate member 20 wherein the user will superpose their feet onto the upper surface of the foot plate member 20. The foot plate member 20 is provided with feet diagram outlines imprinted thereon wherein the user will align their feet with the feet diagram to ensure accurate capture of data. In step 305, the user will be provided instructions via the graphical display unit 15 via a graphical status ring wherein the data mapping progress is displayed to the user. During data acquisition, the status ring indicator 17 is displayed to the user wherein the user must apply pressure to the forefoot region of their feet. The user must maintain this position for three seconds in order to allow sufficient data mapping of the forefoot region of the user's feet. It should be understood within the scope of the present invention that the time for data mapping could vary and be more or less than three seconds.

For the aforementioned movements required for data mapping of the user's foot, it should be understood that during all requested movements the status ring indicator 17 is displayed to the user to ensure the desired pressure is executed as needed to acquire the data mapping of the user's foot. In step 309, the kiosk 10 provides instructions via the graphical display unit 15 for the user to lift their left foot so as to be standing only on the right foot. Step 311, the user is provided an update via the status ring indicator 17. As with other steps discussed herein, the user is required to hold the requested movement for a specified time period. Step 313, the user will return the left foot back to be superposed on the upper surface of the foot plate member 20. In step 315, the user is provided instructions via the graphical display unit 15 to raise their right foot so as to be standing solely on their left foot. Step 317, the user is applies pressure biased to the forefoot region of the right foot wherein the data mapping progress is graphically displayed on the status ring indicator 17. Ensuing holding the immediately aforementioned position, the user returns their right foot to the foot plate member 20. In step 319, the controller 4 of the kiosk 10 begins compilation of the foot mapping data acquired in the aforementioned steps. The controller 4 is a conventional computing processor having the necessary electronics to receive, store, transmit and manipulate data. It should be understood within the scope of the present invention that the foot mapping data acquired contains a multitude of measurements. By way of example but not limitation, the data acquired includes measurements such as the arch height distance, lateral arch height distance, heel depth, inside heel width, lateral flange distance, overall length and forefoot width.

In step 321, the graphical display unit 15 provides a graphical foot map of the user's foot to the user wherein the graphical foot map results from the data acquisition discussed herein. Step 323, during display of the graphical foot map, the user will be shown the identified pressure points on the bottom of their feet as identified during the data acquisition process of the foot mapping. It should be understood within the scope of the present invention that the pressure points could be displayed to the user utilizing alternate techniques such as but not limited to color coding. In step 325, the user has displayed on the graphical display unit 15 the type of orthotic insert 50 that best accommodates their feet based on the foot mapping data thereof. In the preferred embodiment of the present invention, the orthotic apparatus and method 100 includes providing an inventory of fourteen different styles of orthotic inserts 50 that accommodate the user's feet. Furthermore, it should be understood within the scope of the present invention that the orthotic insert 50 includes alternate durometer readings (elasticity) across the three aforementioned regions of the orthotic insert 50 if required.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. A method of determining a proper orthotic insert for feet of the user wherein the method comprises the steps of:
providing a kiosk, said kiosk having a base member, said base member having a foot plate member, said foot plate member having a multitude of sensors embedded therein, said kiosk having housing extending upward from said base member, said housing being comprised of a plurality of walls forming an interior volume, said interior volume having disposed therein a controller, said controller having electronics operable to receive, store, transmit and manipulate data, said kiosk having a graphical display unit in the housing;
activating the kiosk, wherein a user activates the kiosk for performance of data mapping of the feet of the user;
engaging the foot plate member, wherein the user superposes their feet on the foot plate member;
initiating a mapping data scan of the user's feet, wherein the multitude of sensors provide scanning of the user's feet;
manipulating a position of the user's feet, wherein the feet of the user have a position thereof changed at least once during the mapping data scan;
compiling data points from the mapping data scan, wherein the controller manipulates the data points acquired during the mapping data scan to produce a foot map, said foot map display to the user on the graphical display unit;
providing guidance to the user on a proper fitting orthotic, wherein the proper fitting orthotic is provided to the user from an inventory thereof.

2. The method of determining a proper orthotic insert for the feet of the user as recited in claim 1, wherein the mapping data scan includes capture and recording of the following measurements of the user's foot: arch height distance, lateral arch height distance, heel depth, inside heel width, lateral flange distance, overall length and forefoot width.

3. The method of determining a proper orthotic insert for the feet of the user as recited in claim 2, and further including a step of requesting a user to lift a left foot so as to have the user stand on a right foot only.

4. The method of determining a proper orthotic insert for the feet of the user as recited in claim 3, and further including the step of requesting a user to apply pressure to a forefoot region of their feet.

5. The method of determining a proper orthotic insert for the feet of the user as recited in claim 4, and further including a step of providing a status ring indicator, said status ring indicator updated upon a user applying pressure to their feet properly.

6. The method of determining a proper orthotic insert for the feet of the user as recited in claim 5, wherein said inventory of orthotic inserts comprises fourteen different inserts.

7. The method of determining a proper orthotic insert for the feet of the user as recited in claim 6, and further including a step of requesting a user to lift a right foot so as to have the user stand on a left foot only.

8. The method of determining a proper orthotic insert for the feet of the user as recited in claim 7, and further including a step of requesting the user to to apply pressure to the forefoot region of their feet during the mapping data scan.

9. The method of determining a proper orthotic insert for the feet of the user as recited in claim 8, wherein the base member further includes side rail members, said side rail members being located on opposing sides of the base member and extending upward therefrom.

10. The method of determining a proper orthotic insert for the feet of the user as recited in claim 9, wherein the orthotic further includes a plurality of support rib members, said support rib members being integrally formed in an arch region of the orthotic.

11. The method of determining a proper orthotic insert for the feet of the user as recited in claim 10, wherein at least a portion of the orthotics in the inventory have a heel aperture, said heel aperture being bored through the orthotic in a heel region of the orthotic.
